(19) United States
(12) Patent Application Publication
(10) Pub. No.: US 2026/0168999 A1
(43) Pub. Date: Jun. 18, 2026

(54) METHOD OF DETECTING CANCER BIOMARKER CONCENTRATION OR DETECTING EXOSOME CONCENTRATION OF IN VITRO BIOLOGICAL SAMPLE WITH MAGNETIC JANUS PARTICLE CONJUGATED WITH DETECTING ANTIBODY

(71) Applicant: NATIONAL CHENG KUNG UNIVERSITY, Tainan City (TW)

(72) Inventors: Han-Sheng CHUANG, Tainan City (TW); Yu-Hsuan CHOU, Kaohsiung City (TW); Dhrubajyoti DAS, Tainan City (TW)

(21) Appl. No.: 19/193,920

(22) Filed: Apr. 29, 2025

(30) Foreign Application Priority Data
Dec. 13, 2024 (TW) 113148798

(51) Int. Cl. G01N 33/574; G01N 21/63; G01N 33/543; G01N 33/573

(52) U.S. Cl. CPC G01N 33/5759; G01N 21/63; G01N 33/54333; G01N 33/573; G01N 2333/70585; G01N 2333/70596; G01N 2333/96419

(57) ABSTRACT

A method of detecting a cancer biomarker concentration of an in vitro biological sample with a magnetic Janus particle conjugated with a detecting antibody (conjugated MJP) includes providing a solution including the conjugated MJP; adding an in vitro biological sample including exosome to the solution to form a complex, in which the in vitro biological sample is a standard sample with a known concentration or a test sample with a test concentration; performing a treatment including an excited light and an alternative magnetic field at a plurality of driving frequencies to the complex to obtain an SNR-driving frequency curve of the complex in the standard sample or the test sample; obtaining a cut-off frequency from the SNR-driving frequency curve of the standard sample or the test sample; and establishing a standard curve from the cut-off frequency of the standard sample and obtain test concentration of the test sample.

### CROSS-REFERENCE TO RELATED APPLICATION

This application claims priority to Taiwan Application Serial Number 113148798, filed Dec. 13, 2024, which is herein incorporated by reference in its entirety.

### BACKGROUND

### Field of Invention

The present invention relates to a method of detecting an in vitro biological sample. More particularly, the present invention relates to a method of detecting a cancer biomarker concentration of an in vitro biological sample with a magnetic Janus particle conjugated with a detecting antibody.

### Description of Related Art

At the early stage of cancer, the symptoms are not noticeable, or there are even no symptoms, so people may easily neglect the early stage of cancer. The current approaches to facilitate detecting cancer in the early stage include tissue biopsy, DNA quantification, polymerase chain reaction (PCR hereafter), and/or proteomic biomarkers. However, tissue biopsy is invasive, time-consuming, and high-cost. The technical demands of DNA quantification and PCR are high, and DNA quantification and PCR are time-consuming. Thereafter, the proteomic biomarkers require complex analysis and high cost.

Take oral cavity cancer as an example. Oral cavity cancer refers to the cancer that develops in any part that makes up the oral cavity. A global statistic for cancer in 2022 shows that oral cavity cancer is the 16^{th} most common cancer and 95% of oral cavity cancer results from oral squamous cell carcinoma (OSCC). Oral potentially malignant disorders (OPMD) such as leukoplakia, erythroplakia, erythroleukoplakia, oral submucous fibrosis (OSF), palatal lesions of reverse cigar smoking, and/or oral lichen planus are subtle and lack accurate classification as well as identification. Moreover, histopathology detection is challenging. However, there are insufficient biomarkers for oral cavity cancer diagnosis, and thus oral cavity cancer may be confused with other diseases at the early stage.

In view of the above, it is necessary to provide a method of detecting a cancer biomarker concentration of an in vitro biological sample with a magnetic Janus particle conjugated with a detecting antibody to solve the aforementioned problems.

### SUMMARY

According to one aspect of the present invention, a method of detecting a cancer biomarker concentration of an in vitro biological sample with a magnetic Janus particle conjugated with a detecting antibody is provided. First, a solution is provided, in which the solution includes the magnetic Janus particle conjugated with a detecting antibody. The magnetic Janus particle includes a fluorescence core and a metal hemisphere shell. The metal hemisphere shell covers a hemisphere surface of the fluorescence core, and the metal hemisphere shell includes a nickel layer. The detecting antibody conjugates on a surface of the metal hemisphere shell, in which a number ratio of the detecting antibody and the magnetic Janus particle is (150 to 1500000):1. Next, the in vitro biological sample is added to the solution, in which the in vitro biological sample includes a cancer biomarker, the detecting antibody binds to the cancer biomarker, the magnetic Janus particle conjugated with the detecting antibody forms a complex, the in vitro biological sample is a standard sample including the cancer biomarker with a known concentration or a test sample originating from a test subject, and the cancer biomarker includes an exosome. Then, a treatment is performed on the complex to obtain at least one signal-to-noise ratio (SNR hereafter)-driving frequency curve of the complex, in which the treatment includes applying an excited light and an alternative magnetic field at a plurality of driving frequencies to the complex. A magnetic field intensity of the alternative magnetic field is 0.1 mG to 500 mG, and each of the plurality of the driving frequencies is bigger than 1 Hz to 50 Hz. The at least one SNR-driving frequency curve obtained from the standard sample is a first SNR-driving frequency curve, and the at least one SNR-driving frequency curve obtained from the test sample is a second SNR-driving frequency curve. Thereafter, a first cut-off frequency and a second cut-off frequency are obtained from the first SNR-driving frequency curve and the second SNR-driving frequency curve, respectively, with an SNR of bigger than 1 and smaller than 5 as a standard. Next, a test concentration of the test sample is obtained from the second cut-off frequency according to the standard curve established by the first cut-off frequency corresponding to the known concentration.

According to the method of the aforementioned aspect, the cancer biomarker can be used to facilitate anticipating a cancer, and the cancer can include at least one of an oral cavity cancer, a colon cancer, a pancreatic cancer, a renal cancer, a bladder cancer, a breast cancer, a prostate cancer, a blood cancer, and a cartilage tumor.

According to the method of the aforementioned aspect, the in vitro biological sample can originate from at least one of a saliva, an oral mucosa, an intestinal juice, a urine, a breast milk, a semen, blood, a tissue fluid, and a synovial fluid.

According to the method of the aforementioned aspect, the in vitro biological sample can be obtained by subjecting an exosome isolation treatment.

According to the method of the aforementioned aspect, the detecting antibody can include at least one of a CD63 antibody, a CD44 antibody, a CD81 antibody, an ADAM10 antibody, and an MMP14 antibody.

According to the method of the aforementioned aspect, the metal hemisphere shell can further include a silver layer, and the silver layer can be disposed between the nickel layer and the hemisphere surface of the fluorescence core.

According to the method of the aforementioned aspect, the metal hemisphere shell can further include a gold layer disposed on the nickel layer.

According to the method of the aforementioned aspect, a wavelength of the excitation light can be 530 nm to 550 nm.

According to the method of the aforementioned aspect, the method of detecting the cancer biomarker concentration of the in vitro biological sample with the magnetic Janus particle conjugated with the detecting antibody can further include performing a pre-treatment on the complex before the treatment, in which the pre-treatment can include applying the excitation light and the alternative magnetic field at a testing frequency to the complex, and the testing frequency can be smaller than each of the plurality of driving frequencies.

According to the method of the aforementioned aspect, the method of detecting the cancer biomarker concentration of the in vitro biological sample with the magnetic Janus particle conjugated with the detecting antibody can further include performing a reaction step after the in vitro biological sample is added to the solution at 500 rpm to 1000 rpm for 20 minutes to 60 minutes.

According to the method of the aforementioned aspect, the standard curve can be obtained by a linear regression analysis.

According to the method of the aforementioned aspect, the standard sample can include a standard stock solution and a standard diluted solution obtained by diluting the standard stock solution at a specific dilution ratio.

According to the method of the aforementioned aspect, an area of the hemisphere surface covered by the metal hemisphere shell can account for 40% to 60% of a surface of the fluorescence core.

According to another aspect of the present invention, a method of detecting an exosome concentration of an in vitro biological sample with a magnetic Janus particle conjugated with a detecting antibody is provided. First, a solution is provided, in which the solution includes the magnetic Janus particle conjugated with the detecting antibody. The magnetic Janus particle includes a fluorescence core and a metal hemisphere shell. The metal hemisphere shell covers a hemisphere surface of the fluorescence core, and the metal hemisphere shell includes a silver layer, a nickel layer covering the silver layer, and a gold layer covering the nickel layer. The detecting antibody conjugates on a surface of the metal hemisphere shell, in which a number ratio of the detecting antibody and the magnetic Janus particle is (150 to 1500000):1. Next, the in vitro biological sample is added to the solution, in which the in vitro biological sample includes an exosome, the detecting antibody binds to a transmembrane protein on the exosome, the magnetic Janus particle conjugated with the detecting antibody forms a complex, and the in vitro biological sample is a standard sample including the exosome with a known concentration or a test sample originating from a test subject. Next, a treatment is performed on the complex to obtain at least one SNR-driving frequency curve of the complex. The treatment includes applying an excited light and an alternative magnetic field at a plurality of driving frequencies to the complex. A magnetic field intensity of the alternative magnetic field is 0.1 mG to 500 mG. Each of the plurality of the driving frequencies is bigger than 1 Hz to 50 Hz. The at least one SNR-driving frequency curve obtained from the standard sample is a first SNR-driving frequency curve, and the at least one SNR-driving frequency curve obtained from the test sample is a second SNR-driving frequency curve. Thereafter, a first cut-off frequency and a second cut-off frequency is obtained from the first SNR-driving frequency curve and the second SNR-driving frequency curve, respectively, with an SNR of bigger than 1 and smaller than 5 as a standard. Moreover, a standard curve is established by the first cut-off frequency corresponding to the known concentration, and a test concentration of the test sample is obtained from the second cut-off frequency according to the standard curve.

According to the method of the aforementioned aspect, the transmembrane protein can include at least one of a CD63, a CD44, a CD81, an ADAM10, and an MMP14.

### BRIEF DESCRIPTION OF THE DRAWINGS

The invention can be more fully understood by reading the following detailed description of the embodiment, with reference made to the accompanying drawings as follows:

FIG. 1 is a flow diagram illustrating the method of detecting a cancer biomarker concentration of an in vitro biological sample with a magnetic Janus particle conjugated with detecting antibodies according to an embodiment of the present invention.

FIG. 2 is a schematic diagram illustrating a magnetic Janus particle conjugated with detecting antibodies according to an embodiment of the present invention.

FIG. 3 is a cross-sectional schematic diagram illustrating a magnetic Janus particle conjugated with detecting antibodies according to an embodiment of the present invention.

FIGS. 4A and 4B are schematic diagrams illustrating a complex 400A of a standard sample and a complex 400B of a test sample, respectively, according to an embodiment of the present invention.

FIGS. 5A and 5B are a curved graph 500A and a curved graph 500B illustrating the computer simulation of the fluorescence intensity of the magnetic Janus particle (MJP) at different times when each of the driving frequencies is 3 Hz (FIG. 5A) and 22 Hz (FIG. 5B), respectively, according to some embodiments of the present invention.

FIGS. 6A and 6B illustrate an SNR-driving frequency curved graph 600A and an SNR-driving frequency curved graph 600B at 3 Hz (FIG. 6A) and 22 Hz (FIG. 6B), respectively, according to some embodiments of the present invention.

FIGS. 7A to 7D illustrate the simulated SNR-driving frequency curved graphs under different viscosities (FIG. 7A), different magnetic field intensities (FIG. 7B), different equivalent particle sizes of the MJP (FIG. 7C), and different particle sizes of the hydrodynamic volumes of the MJP (FIG. 7D).

FIG. 8 is a curved graph 800 illustrating the changes in the fluorescence intensity of the MJPs over time without an external magnetic field.

FIG. 9 is a curved graph 900 illustrating the fluorescence intensity of the MJPs over time under an external magnetic field whose polarity is fixed.

FIGS. 10A and 10B are a curved graph 1000A and a curved graph 1000B illustrating the fluorescence intensity of the MJPs over time as each of the driving frequencies is 3 Hz (FIG. 10A) and 22 Hz (FIG. 10B).

FIG. 11A illustrates an SNR-driving frequency curved graph of the MJPs with different equivalent particle sizes according to some embodiments of the present invention.

FIG. 11B is a bar graph 1100 illustrating the cut-off frequencies of the SNR-driving frequency curves of the MJPs having different equivalent particle sizes according to the SNR-driving frequency curves in FIG. 11A.

FIG. 12A illustrates an SNR-driving frequency curved graph of the MJPs at different voltage amplitudes according to some embodiments of the present invention.

FIG. 12B is a bar graph 1200 illustrating the cut-off frequencies of the SNR-driving frequency curves of the MJPs at different voltage amplitudes according to the SNR-driving frequency curve in FIG. 12A.

FIG. 13A illustrates an SNR-driving frequency curved graph of the MJPs with different distances away from the electromagnet according to some embodiments of the present invention.

FIG. 13B is a bar graph 1300 illustrating the cut-off frequency of the SNR-driving frequency curve of the MJPs with different distances away from the electromagnet according to FIG. 13A.

FIG. 14A illustrates an SNR-driving frequency curved graph of the MJPs at different viscosities according to some embodiments of the present invention.

FIG. 14B is a bar graph 1400 illustrating the cut-off frequency of the SNR-driving frequency curve of the MJPs at different viscosities according to FIG. 14.

FIG. 15A illustrates the SNR-driving frequency curves in different groups according to different groups of the present invention.

FIG. 15B is a bar chart 1500 illustrating the cut-off frequencies of the SNR-driving frequency curves in different groups according to FIG. 15A.

FIG. 16 is a scatter graph illustrating the driving frequencies corresponding to the logarithm of the exosome concentration of Example Groups 1 to 3 according to FIG. 15B.

### DETAILED DESCRIPTION

As shown above, the present invention relates to a method of detecting a cancer biomarker concentration of an in vitro biological sample with a magnetic Janus particle conjugated with detecting antibodies. In the method, the magnetic Janus particle conjugated with detecting the antibodies and an alternative magnetic field at a plurality of the driving frequencies are used to obtain the cut-off frequency, and the test concentration is obtained from the cut-off frequency of the test sample according to the standard curve established by using the known concentration of a standard sample and the cut-off frequency. The method can shorten the detecting time as well as decrease the lower limit of the volume and/or concentration of a sample, thereby increasing the detecting concentration range.

The term “in vitro biological sample” herein can refer to a sample isolated from the body of a subject. In some embodiments, the in vitro biological sample can originate from a subject's body fluid including a cancer biomarker, for example. In some specific examples, the body fluid can include but not limited to at least one of a saliva, an oral mucosa, an intestinal juice, a urine, a breast milk, a semen, a blood, a tissue fluid, and a synovial fluid. The in vitro biological sample can be a standard sample or a test sample, for example. In some specific examples, the standard sample originates from a healthy subject or a sample with a known concentration. In some specific examples, the test sample originates from the sample of a test subject. In some embodiments, the test subject can include but not limited to a subject suspected of having cancer and/or a subject undergoing a health checkup.

The aforementioned method of detecting the cancer biomarker concentration of the in vitro biological sample with the magnetic Janus particle conjugated with the detecting antibodies can be used to facilitate anticipating cancer, e.g., anticipating early-stage cancer. The term “early-stage cancer” herein refers to the early stage of cancer development. In view of tumor types, the early-stage cancer refers to a solid tumor whose abnormal cells or tumors are still in situ and have not spread to nearby tissue, or refers to the blood cancer whose blast cells appear in small amounts in blood and/or bone mass, or a lymphoma that is restricted only in a lymphatic zone. For example, early-stage oral cavity cancer refers to the abnormal cells or a tumor smaller than 2 cm of the oral epithelium that has not invaded in the lymph node. The cancer can include but not limited to at least one of an oral cavity cancer, a pancreatic cancer, a colon cancer, a renal cancer, a bladder cancer, a breast cancer, a prostate cancer, a blood cancer, and a cartilage tumor. In some embodiments, the oral cavity cancer can be an oral squamous-cell carcinoma, for example.

The “cancer biomarker” herein refers to a biomarker that can differentiate tumor cells from normal cells. Studies have shown that the exosome plays an important role during cancer development. The term “exosome” herein refers to vesicles with a diameter of 30 nm to 150 nm excreted by a cell, and the exosome belongs to a subset of extracellular vesicles (EV). The biogenesis of the exosome involves an endosomal system. In the system, the early endosomes are formed by endocytosis and are subsequently matured into a late endosome or a multivesicular body (MVB hereafter) with intraluminal vesicles (ILVs hereafter). After the late endosome or the MVB fuses with the plasma membrane, the ILVs can release and form the exosomes. The exosome includes a hydrophilic core and a phospholipid bilayer structure surrounding the hydrophilic core. The hydrophilic core includes the genetic material (e.g., DNA, mRNA, and microRNA), protein, and/or metabolites so that the exosome can play an important role in intercellular communication, thereby influencing many physiological reactions such as immune response, tissue repair, stem cell maintenance, and/or central nervous system communication.

Studies have found that the exosome involves in the pathological processes of cardiovascular disease, neurodegeneration, cancer, and/or inflammation. For example, the exosome produced by a cancer cell is different from that produced by a normal cell in quantity, cargo of genetic materials, and/or transmembrane proteins. Therefore, it can facilitate anticipating the risk of early-stage cancer by detecting the exosome concentration in an in vitro biological sample by the antigen on the exosome.

Since there is a specific antigen on an exosome, the exosome concentration can be detected by using a detecting antibody. The term “detecting antibody” herein refers to an immunoglobulin that can specifically bind with the antigen on the exosome. In some embodiments, the antigen can be the transmembrane protein located on the exosome. In some specific examples, the aforementioned transmembrane protein can include but not limited to a CD63, a CD44, a CD81, an ADAM10, and/or an MMP14. In some specific examples, the detecting antibody can include but not limited to a CD63 antibody, a CD44 antibody, a CD81 antibody, an ADAM10 antibody, and an MMP14 antibody, for example.

The term “magnetic Janus particle (MJP)” herein refers to a particle whose two hemispheres have different properties, in which one of the hemispheres of the particle can generate fluorescent with a specific wavelength under an excitation light with a specific wavelength. The other one of the hemispheres of the particle is covered by metal and will not generate fluorescent and can be magnetized by an external magnetic field so that the magnetic Janus particle can specifically bind with the exosome and rotate back and forth corresponding to an alternative magnetic field. The term “magnetic Janus particle conjugated with a detecting antibody” is the MJP having at least one detecting antibody conjugated on a surface of the metal.

The term “complex” herein is formed by a magnetic Janus particle conjugated with a detecting antibody as the detecting antibody binds with the exosome. Generally, the higher the exosome concentration of the in vitro biological sample is, the more the exosomes to which the detecting antibody binds, and the bigger the hydrodynamic volume of the complex is. The term “hydrodynamic volume” herein refers to the volume of the magnetic Janus particle conjugated with the detecting antibody accounts as it moves (including rotation) in a solution after the magnetic Janus particle conjugated with the detecting antibody binds to the exosome with the detecting antibody. It is noted that the particle size of the magnetic Janus particle conjugated with the detecting antibody is referred to as “an equivalent particle size” herein when the detecting antibody does not bind to an exosome.

The term “alternative magnetic field” herein refers to a magnetic field that the polarity can switch periodically. In some embodiments, the alternative magnetic field is generated by using an electromagnet introduced with an electric current that changes periodically. In some specific examples, the electric current that changes periodically can be the alternating current or the direct current switched on and off periodically, for example. In some specific examples, the voltage waveform of the electric current that changes periodically can be a square wave, for example. Since the polarity of the alternative magnetic field switches at a frequency that is determined by the changing period of the electric current, the frequency at which the polarity of the alternative magnetic field switches is referred to as “driving frequency”.

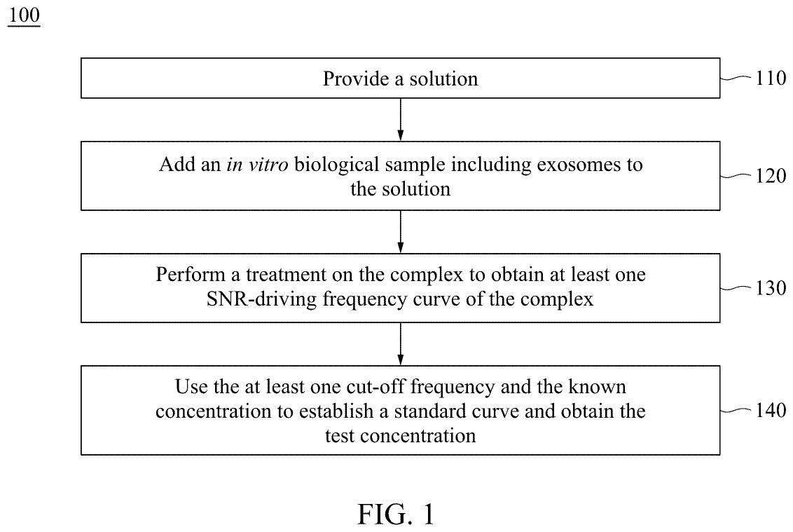

The following describes the various steps of the method 100 of detecting a cancer biomarker concentration of an in vitro biological sample with a magnetic Janus particle conjugated with detecting antibodies with FIG. 1, which is a flow diagram illustrating the method 100 of detecting a cancer biomarker concentration of an in vitro biological sample with a magnetic Janus particle conjugated with detecting antibodies according to an embodiment of the present invention.

According to Operation 110 shown in FIG. 1, provide a solution. The solution includes magnetic Janus particles conjugated with detecting antibodies and a solvent used to suspend the magnetic Janus particles conjugated with the detecting antibodies. In some embodiments, the solvent can include but not limited to a salt buffer without surfactant to benefit the addition of the solution to the in vitro biological sample including exosome subsequently. In some specific examples, the buffer can include but not limited to phosphate buffered saline (PBS hereafter), hydroxyethylpiperazine ethane sulfonic acid (HEPES) buffer solution, 2-morpholinoethanesulphonic acid (MES) buffer solution, (3-(N-morpholino) propanesulfonic acid, MOPS) buffer solution, and/or tris(hydroxymethyl)aminomethane buffered saline (TBS) solution. The amount of the magnetic Janus particles conjugated with the detecting antibodies can be 1×10⁶ particles/mL to 1×10¹⁰ particles/mL, for example, so as to increase the detection sensitivity but prevent the interval between the magnetic Janus particles conjugated with the detecting antibodies from being too short, or else the van der Waals force cannot overcome the electrostatic repulsion, resulting in the magnetic Janus particles conjugated with the detecting antibodies attracting each other and gathering.

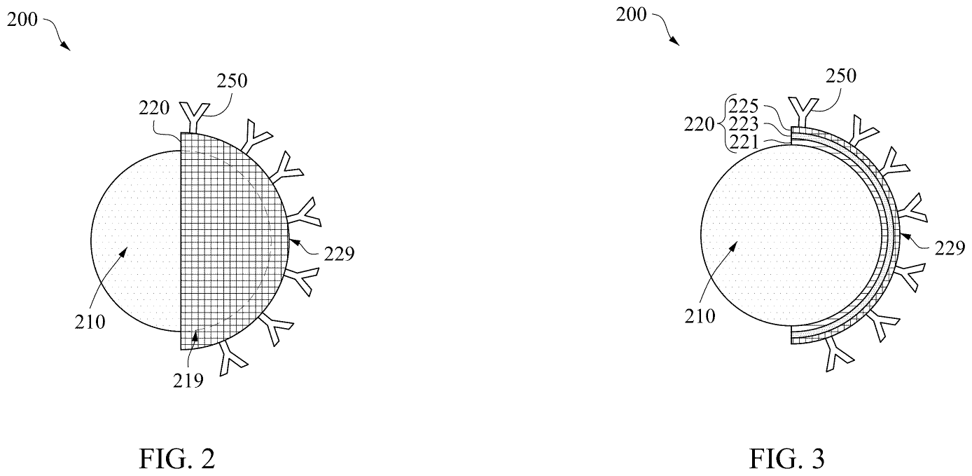

Reference is made to FIG. 2, which is a schematic diagram illustrating a magnetic Janus particle 200 conjugated with detecting antibodies 250 according to an embodiment of the present invention. As shown in FIG. 2, the magnetic Janus particle 200 conjugated with the detecting antibodies 250 includes a fluorescence core 210, a metal hemisphere shell 220, and detecting antibodies 250. In some embodiments, the particle size of the fluorescence core 210 is smaller than or equal to 10.0 μm, e.g., 0.1 μm to 10.0 μm, so that the magnetic Janus particle 200 conjugated with the detecting antibodies 250 obtained therefrom has a higher sensitivity. In some embodiments, the fluorescence core 210 can be a red fluorescent polystyrene that can emit light with a wavelength of 575 nm to 675 nm under the excitation of green light (the wavelength is 530 nm to 550 nm).

The metal hemisphere shell 220 covers a hemisphere surface 219 of the fluorescence core 210. In some embodiments, the area of the hemisphere surface 219 covered by the metal hemisphere shell 220 accounts for 40% to 60% of that of the fluorescence core 210. The metal hemisphere shell 220 can include but not limited to a ferromagnetic material, so that the metal hemisphere shell 220 is magnetized under the external magnetic field and becomes magnetic. In some specific examples, the ferromagnetic materials can include but not limited to iron, cobalt, and/or nickel. In some specific examples, the metal hemisphere shell 220 can include but not limited to nickel. In some embodiments, the metal hemisphere shell 220 can include but not limited to a nickel layer 223. In some embodiments, the thickness of the nickel layer can be 10 nm to 25 nm, for example, e.g., 15 nm to 25 nm, to generate a magnetic moment sufficient to make the magnetic Janus particle 200 conjugated with the detecting antibodies 250 rotate in the alternative magnetic field.

Reference is made to FIG. 3, which is a cross-sectional schematic diagram illustrating a magnetic Janus particle 200 conjugated with the detecting antibody 250 according to an embodiment of the present invention. As shown in FIG. 3, the metal hemisphere shell 220 includes multiple metal layers. In some embodiments, the multiple metal layers can include but not limited to a silver layer 221, the nickel layer 223, and a gold layer 225 from inside to outside. In the embodiment, the silver layer 221 is disposed between the nickel layer 223 and the hemisphere surface 219 covered by the metal hemisphere shell 220, so that the portion of the metal hemisphere shell 220 connecting different magnetic Janus particle 200 can be stripped by sonication during the process of making the magnetic Janus particle 200. In some embodiments, the thickness of the silver layer 221 can be 1 nm to 15 nm, for example, e.g., 5 nm to 15 nm.

In some embodiments, the metal hemisphere shell 220 can selectively include the gold layer 225, in which the gold layer 225 is disposed on the nickel layer 223 to facilitate the fixation of the detecting antibody 250 on the surface 229 of the metal hemisphere shell 220 subsequently. In some embodiments, the thickness of the gold layer 225 can be 1 nm to 15 nm, for example, e.g., 5 nm to 10 nm.

The metal hemisphere shell 220 can be formed by the method of physical vapor deposition (PVD hereafter). In some embodiments, the physical vapor deposition can include but not limited to ion plating PVD, sputtering PVD, and/or evaporation PVD. In some specific examples, the evaporation PVD can include but not limited to resistive PVD, inductive PVD, laser PVD, and/or e-bean PVD. In the examples that the metal hemisphere shell 220 includes multiple-metal layers as shown in FIG. 3, and the silver layer 221, the nickel layer 223, and the gold layer 225 can be disposed sequentially.

The detecting antibody 250 can be fixed on the surface 229 of the metal hemisphere shell 220. The method to fix the detecting antibody 250 can form a covalent bond between the detecting antibody 250 and the surface 229 of the metal hemisphere shell 220. The covalent bond can be an amide bond formed by using carbodiimide crosslinker chemistry. A number ratio of the detecting antibodies 250 and the magnetic Janus particle 200 can be (150 to 1500000):1, for example. The number ratio is the upper limit of the detecting antibodies 250 that can be carried by the metal hemisphere shell 220 of the magnetic Janus particle 200. The number ratio relates to the surface area of the metal hemisphere shell 220, and the surface area of the metal hemisphere shell 220 is proportional to the square of the particle size of the magnetic Janus particle 200. In some specific examples, when the particle size of the magnetic Janus particle 200 is 1 μm, the number ratio of the detecting antibodies 250 and the magnetic Janus particle 200 can be (10000 to 16000):1, for example.

As shown in FIG. 1, followed by Operation 110, Operation 120 is performed to add an in vitro biological sample including exosomes to the solution. In some embodiments, the in vitro biological sample can be obtained by subjecting the body fluid to a cancer biomarker isolation treatment. In some specific examples, the cancer biomarker isolation treatment can include but not limited to an exosome isolation treatment, e.g., ultracentrifugation, to isolate the exosome. During Operation 120, at least one of the magnetic Janus particles conjugated with the antibodies in the solution can form a complex by binding at least one exosome with at least one detecting antibody. In some embodiments, after the in vitro biological sample is added to the solution, a reaction step can be selectively performed at room temperature (15° C. to 35° C.), 500 rpm to 1000 rpm for 20 minutes to 60 minutes.

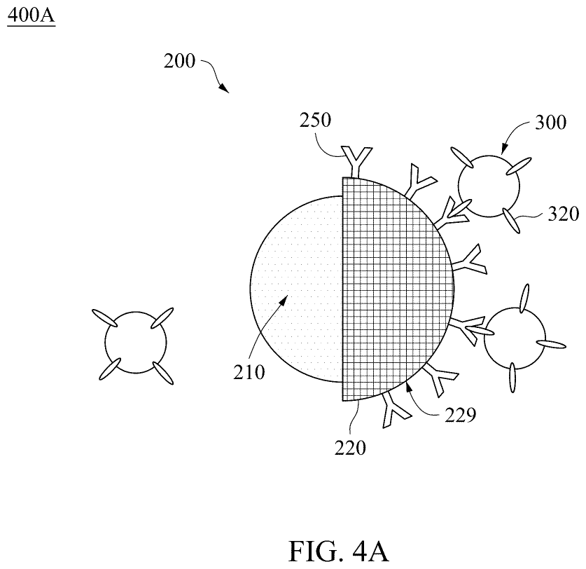

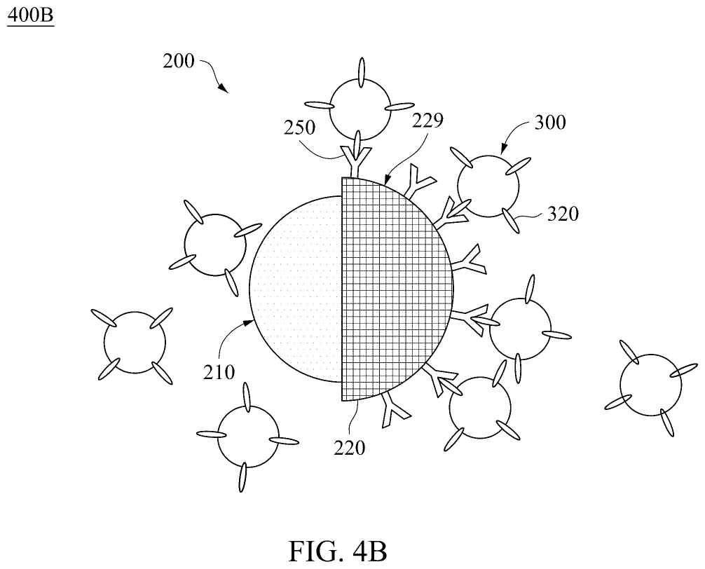

FIGS. 4A and 4B are schematic diagrams of a complex 400A of a standard sample and a complex 400B of a test sample, respectively, according to an embodiment of the present invention. FIGS. 4A and 4B are used to illustrate a test sample whose concentration of the exosome 300 is bigger than the concentration of the exosome 300 of the standard sample, e.g., an example that has a risk of early-stage cancer and does not mean to put constraint on the present invention. In other embodiments, the test sample whose concentration of the exosome 300 can be smaller than the concentration of the exosome 300 of the standard sample, i.e., the test sample has no risk of early-stage cancer.

As shown in FIG. 4A, the transmembrane proteins 320 of the exosomes 300 in the standard sample can bind to the at least one of the detecting antibodies 250 on the surface 229 of the metal hemisphere shell 220 of the magnetic Janus particle 200 conjugated with the detecting antibodies 250, thereby forming the complex 400A. Moreover, as shown in FIG. 4B, since the concentration of the exosome 300 in the test sample is higher, the complex 400B binds to more exosomes 300, resulting in a bigger hydrodynamic volume.

Next, Operation 120 is followed by Operation 130 to perform a treatment on the complex to obtain an SNR-driving frequency curve of the complex. The SNR-driving frequency curve obtained from the standard sample is referred to as a first SNR-driving frequency curve, and the SNR-driving frequency curve obtained from the test sample is referred to as a second SNR-driving frequency curve.

The aforementioned treatment includes applying an excitation light and an alternative magnetic field at a plurality of driving frequencies to the complex. Specifically, Operation 130 is performed with a fluorescence microscope equipment. In detail, an electromagnet is disposed on the stage of the fluorescence microscope, and after the electromagnet is introduced with a direct current whose current direction switches periodically, the magnetic field lines in the electromagnet are perpendicular to the stage, and the polarity switches along with the driving frequencies. The light source of the excitation light is disposed under the fluorescence microscope to excite the sample on the stage to generate fluorescence. An image-capturing device can be disposed in the observation direction, in which the image-capturing device can be a camera, for example. In some specific examples, the camera can include but not limited to a charge-coupled device (CCD) camera and/or a complementary metal oxide semiconductor (CMOS) camera.

The magnetic field intensity of the alternative magnetic field generated by the electromagnet on the magnetic Janus particle conjugated with the detecting antibody can be 0.1 milligauss (mG hereafter) to 500 mG, for example, e.g., 0.3 mG to 10.0 mG, or 0.32 mG to 6.10 mG, to maintain the distance between the microscope stage and the electromagnet benefit the detecting operation, and it is ensured that the magnetic field intensity will not affect the operation of other instruments. If the magnetic field intensity of the alternative magnetic field is too low, the magnetic moment produced by the metal hemisphere shell being magnetized is not sufficient to make the magnetic Janus particle conjugated with the detecting antibody rotate. If the magnetic field intensity of the alternative magnetic field is too high, the operation of other instruments can be negatively affected, and the electromagnet should be very close to the microscope stage, resulting in the operation space being too small to the operate.

As mentioned above, the metal hemisphere shell covers the hemisphere surface of the fluorescence core, in which the metal hemisphere shell can be magnetized by an external magnetic field, so that the complex rotates back and forth along with the driving frequency of the alternative magnetic field, thereby changing the portion of the complex projected to the observation direction. On the other hand, the other surface of the hemisphere of the fluorescence core is exposed by the metal hemisphere shell such that the surface of the hemisphere being exposed can generate fluorescence under the irradiation of the excitation light.

When there is an increase or a decrease in the area of the hemisphere surface of the fluorescence core that is exposed by the metal hemisphere shell and projects to the observation direction, the fluorescence intensity that is generated by the complex and can be observed in the observation direction will increase or decrease consequently. In other words, the bigger the area of the hemisphere surface of the fluorescence core that is exposed by the metal hemisphere shell and projects to the observation direction, the stronger the fluorescence intensity detected by the observation direction. Moreover, when a back-and-forth rotation angle of the complex is smaller than 180°, the area of the hemisphere surface of the fluorescence core that is exposed by the metal hemisphere shell and projects to the observation direction changes regularly, so that the complex can be observed to “blink” in the observation direction. The bigger the back-and-forth rotation angle of the complex, the bigger the amplitude of the fluorescence intensity detected by the observation direction. Formula 1 shows the relationship between the upper limit of the amplitude of the fluorescence intensity, the rotation angle, and other parameters.

$${I}_{m ax} = A \times \frac{{d}_{p}^{2}}{4} \times Δ {θ}_{m ax} [ sin ( 2 π f t + ϕ ) + 1 ] . \qquad ( Formula 1 )$$

In Formula 1, the symbol Iₘₐₓ represents the upper limit of the amplitude of the fluorescence intensity of the complex; the symbol A represents the coefficient; the symbol dₚ represents the equivalent particle size of a magnetic Janus particle, i.e., the diameter of the magnetic Janus particle not conjugated with the detecting antibody and not bound with an exosome; the symbol Δθₘₐₓ represents the upper limit of back-and-forth rotation angle of the complex in the alternative magnetic field, and the upper limit is smaller than or equal to IT; the symbol f represents the driving frequency of the alternative magnetic field; the symbol t represents time; and the symbol ¢ represents phase.

It is noted that as the complex rotates back and forth along with the driving frequency of the alternative magnetic field, the upper limit of the back-and-forth rotation angle of the complex is affected by the equivalent particle size of the magnetic Janus particle, the hydrodynamic volume of the complex, the viscosity of the solution, the magnetic field intensity of the alternative magnetic field, and/or the driving frequency. Reference is made to Formula 2.

$$〈 {Δθ}_{m ax} 〉 = \frac{{χ}^{″} \times {V}_{m \times } {B}^{2}}{κ \times η \times f \times {V}_{H} \times {μ}_{0}} . \qquad ( Formula 2 )$$

In Formula 2, the symbol Δθₘₐₓ represents the upper limit of the back-and-forth rotation angle of the complex, in which Δθₘₐₓ is smaller than or equal to π; the symbol χ″ represents the imaginary part of the magnetic susceptibility that is proportional to the square of the equivalent particle size of the magnetic Janus particle; and the symbol Vₘ represents the volume of the magnetic material (i.e., the nickel layer). It is noted that since the nickel layer is very thin, Vₘ can be considered to be proportional to the square of the equivalent particle size of the magnetic Janus particle; the symbol B represents the magnetic field intensity; the symbol κ represents the shape factor of the magnetic Janus particle; the symbol η represents the viscosity of the solution; the symbol f represents the driving frequency of the alternative magnetic field; the symbol V_{H} represents the hydrodynamic volume of the complex; and the symbol μ₀ represents a permeability of free space.

It is noted that, practically, the upper limit of the amplitude of the fluorescence intensity is represented by an SNR which is the average of ratios of the upper limits of the amplitudes of the fluorescence intensities of a complex to the background values, so as to reduce the interference of the background. An SNR-driving frequency curve can be obtained by correspondingly detecting the upper limits of the amplitudes of the fluorescence intensities of the complex in the alternative magnetic field at the plurality of the driving frequencies. In some embodiments, the SNR-driving frequency curve is an exponential one-phase decay equation. Specifically, the SNR-driving frequency curve is illustrated with the plurality of driving frequencies of the alternative magnetic field as the x-axis and the SNR as the y-axis.

As shown in Formulas 1 and 2, the SNR of a complex is proportional to the upper limit of the back-and-forth rotation angle of the complex in the alternative magnetic field. However, the upper limit of the angle is inversely proportional to the driving frequency of the alternative magnetic field. Therefore, the SNR-driving frequency curve can be fit as an exponential decreasing function. In some embodiments, the exponential decreasing function can be shown as Formula 3, for example.

$$SNR = A \times {e}^{\frac{- t}{τ}} . \qquad ( Formula 3 )$$

The symbol SNR represents SNR, the symbol A represents the initial signal intensity of the SNR when the time is 0, t represents time, and T represents an attenuation coefficient.

As shown in Formula 1 to Formula 3, as the driving frequency increases, the smaller the upper limit of the back-and-forth rotation angle of the complex is, the smaller the upper limit of the amplitude of the fluorescence intensity of the complex is, and the harder the fluorescent signal can be distinguished from the background value, i.e., the SNR approaches 1. Moreover, the extent to which the SNR of the complex decreases as the driving frequency of the alternative magnetic field increases is related to the particle size of the complex, the hydrodynamic volume, and/or the viscosity. Therefore, when applying the alternative magnetic field at a plurality of driving frequencies to the complex, the particle size, the hydrodynamic volume of the complex, and/or the viscosity can be estimated by the change of the SNR of the complex corresponding to the driving frequencies.

Each of the plurality of the aforementioned driving frequencies can be bigger than 1 Hz to 50 Hz, for example, e.g., 3 Hz to 22 Hz. Since it is better for the frames per second (FPS hereafter) of the camera to be at least four times the driving frequency, the recording should be made by a camera that is more advanced if the driving frequency is too high, thereby increasing the cost of image recording. If the driving frequency is too low, the back-and-forth rotation of the complex in an alternative magnetic field is difficult to distinguish from the Brownian motion. Moreover, the amplitude of the fluorescence intensity of the complex has not reached the lower limit, and thus the cut-off frequency cannot be obtained in the subsequent steps. It is noted that in some embodiments in Operation 130, the alternative magnetic field is applied to the complex at a small driving frequency to a bigger one to ensure that the complex can rotate back and forth along with the plurality of the driving frequencies of the alternative magnetic field during the process and to ensure that the reason the amplitude of the fluorescence intensity hardly distinguish from the background value is that the rotation angle of the complex is too small instead of other reasons (e.g., the complex cannot be magnetized and/or projects to the observation direction at a bad angle).

In some embodiments, Operation 130 can selectively include performing a pre-treatment on the complex before applying the alternative magnetic field at the plurality of driving frequencies to the complex to ensure that the complex can rotate back and forth along with the alternative magnetic field. The pre-treatment can include but not limited to applying an excitation light and an alternative magnetic field at a testing frequency to the complex. The testing frequency can be smaller than the plurality of the driving frequencies, for example. In some specific examples, the testing frequency is bigger than 0 Hz and smaller than 2 Hz.

After Operation 130, a first cut-off frequency and a second cut-off frequency are obtained from the first SNR-driving frequency curve and the second SNR-driving frequency curve, respectively, with an SNR of bigger than 1 and smaller than 5 as a standard. It is noted that the cut-off frequency represents the driving frequency when the amplitude of the fluorescence intensity of the complex is hard to distinguish from the background signal. Ideally, the cut-off frequency should be obtained with an SNR of 1 as a standard. However, practically, when the cut-off frequency should is obtained with an SNR of 1 as the standard, it is hard to distinguish the differences caused by different parameters (particle size, hydrodynamic volume, and/or viscosity) since the amplitude of the fluorescence intensity is hard to distinguish from the background signal. Therefore, in some specific examples, the cut-off frequency can be obtained with an SNR of 1 to 3 as standard, for example.

As shown in Formula 1 and Formula 2, the SNR of the complex is proportional to the upper limit of the back-and-forth rotational angle of the complex, the upper limit of the angle is inversely proportional to the hydrodynamic volume of the complex, and the hydrodynamic volume of the complex is positively correlated to the exosome concentration of the in vitro biological sample. Therefore, with the same driving frequency, the higher the exosome concentration of the in vitro biological sample, the smaller the upper limit the back-and-forth rotational angle of the complex and the closer the SNR gets to 1. Thus, the higher the exosome concentration of the in vitro biological sample, the smaller the cut-off frequency.

After Operation 130, Operation 4 is performed to use the at least one cut-off frequency and the known concentration to establish a standard curve and obtain the test concentration. In some embodiments, the standard sample can include but not limited to a standard stock solution including the exosome with a known concentration and standard diluted solutions obtained by diluting the standard stock solution at a specific dilution ratio. The diluted concentration of the standard diluted solutions can be obtained by the known concentration and dilution ratio, and the standard curve can be established by the known concentration of the standard stock solution, the diluted concentration of the standard diluted solution, and the first cut-off frequencies. In some specific examples, the standard curve can be obtained by a linear regression analysis. In some embodiments, the test concentration of the test sample can be obtained from the second cut-off frequency according to the standard curve by interpolation or extrapolation.

It is noted that the result may not be precise if the SNR of the complex is obtained in an alternative magnetic field at a single driving frequency. In the present invention, since the SNR of the complex at a plurality of driving frequencies is measured, and the subsequent process includes a plurality of sampling treatments and statistical analysis to obtain the SNR-driving frequency curve and establish the standard curve, the results can be more precise.

It is noted that the alternative magnetic field used in the method is generated by introducing a current whose direction switches periodically to an electromagnet so that the complex can rotate back and forth at an angle smaller than 180° and can be controlled by the external magnetic field continuously. The alternative magnetic field cannot be replaced by a permanent magnet rotating in a single direction since the rotation of the permanent magnet is controlled by a motor and thus requires larger equipment and more expensive moving parts. Moreover, as the rotational frequency of the permanent magnet gets higher, the complex may not rotate along with the change of the magnetic field and therefore “slip” from the control of the permanent magnet.

Several examples are used below to illustrate the application of the present invention, but they are not used to limit the present invention. Those having ordinary skill in the art of the present invention can make various changes and modifications without departing from the spirit and scope of the present invention.

### Example 1: Computer Simulations of SNR-Driving Frequency Curve of MJP in Different Environments

### 1. Simulating Amplitude of Fluorescence Intensity of MJP in Alternative Magnetic Field at Different Driving Frequencies

The amplitude of the fluorescence intensity of an MJP having an equivalent particle of 1 μm was simulated by computer in an alternative magnetic field at a testing frequency (1 Hz) and a plurality of driving frequencies (3 Hz, 5 Hz, 8 Hz, 10 Hz, 12 Hz, 15 Hz, 18 Hz, 20 Hz, and 22 Hz) under the condition that the magnetic field intensity was 0.01 tesla (T), the solvent was water, and the FPS of camera was 80 Hz.

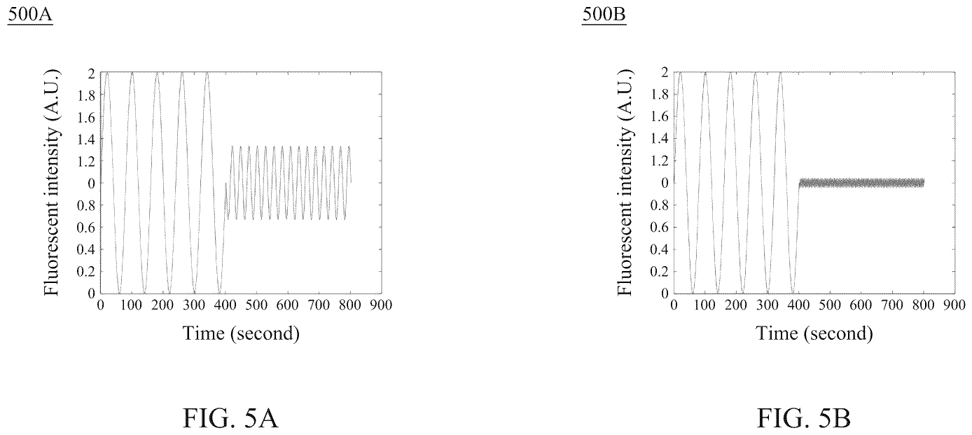

Reference is made to FIGS. 5A and 5B, which are a curved graph 500A and a curved graph 500B illustrating the computer simulation of the fluorescence intensity of the MJP at different times when the each of the plurality of driving frequencies is 3 Hz (FIG. 5A) and 22 Hz (FIG. 5B), respectively, according to some embodiments of the present invention. The x-axis represents time (unit: second), and the y-axis represents fluorescence intensity (unit: arbitrary unit, A.U.).

As shown in FIGS. 5A and 5B, the fluorescence intensities at 0^{th} second to the 400^{th} second are obtained from the MJP in an alternative magnetic field at a testing frequency, and the fluorescence intensities at 401^{th} second to the 800^{th} second are obtained from the MJP in an alternative magnetic field at the corresponding driving frequencies (at 3 Hz in FIG. 5A, and 22 Hz in FIG. 5B). Moreover, the fluorescence intensities of the MJP change periodically, indicating that the MJP rotates back and forth in the alternative magnetic field. In addition, compared to the alternative magnetic field at a testing frequency, the MJP has a smaller amplitude of the fluorescence intensity in the alternative magnetic field at the driving frequencies, and the MJP has a much smaller amplitude of the fluorescence intensity in the alternative magnetic field at the driving frequency of 22 Hz, indicating that the higher the driving frequency, the smaller rotational angle of the complex, and thus the smaller the amplitude of the fluorescence intensity.

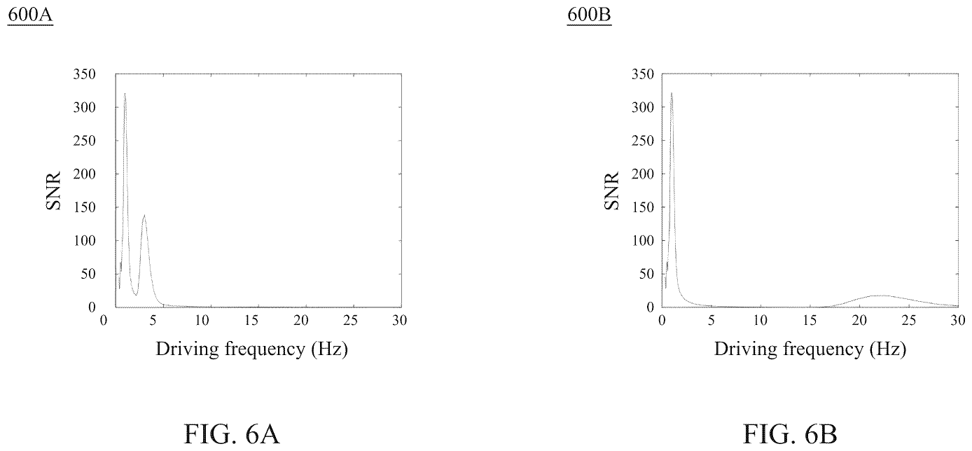

The SNR is obtained by dividing the upper limit of the amplitude of the fluorescence intensity to the background value. An SNR-driving frequency curve of the MJP can be obtained with the driving frequency of the alternative magnetic field as the x-axis and the SNR of the MJP as the y-axis. Reference is made to FIG. 6A and FIG. 6B, which illustrate an SNR-driving frequency curved graph 600A and an SNR-driving frequency curved graph 600B at 3 Hz (FIG. 6A) and 22 Hz (FIG. 6B), respectively, according to some embodiments of the present invention. The x-axis represents the driving frequency (unit: Hz), and the y-axis represents SNR.

As shown in FIGS. 6A and 6B, the higher the driving frequency, the smaller the upper limit of the rotation angle of the MJP. Thus, the MJP has the SNRs at the driving frequency smaller than that at the testing frequency (1 Hz), and the SNR at the driving frequency of 22 Hz is smaller than that at the driving frequency of 3 Hz. It is noted that the simulation results at the driving frequencies of 5 Hz, 8 Hz, 10 Hz, 12 Hz, 15 Hz, 18 Hz, and 20 Hz also show that the SNR decreases (not shown in the drawings) as the driving frequency increases.

### 2. Simulating SNR-Driving Frequency Curve of MJP at Different Conditions

The computer simulation of the SNR-driving frequency curves of the MJP under different conditions was performed by changing the viscosity, the magnetic field intensity, the equivalent particle size of the MJP, and the particle size of the hydrodynamic volume of the MJP.

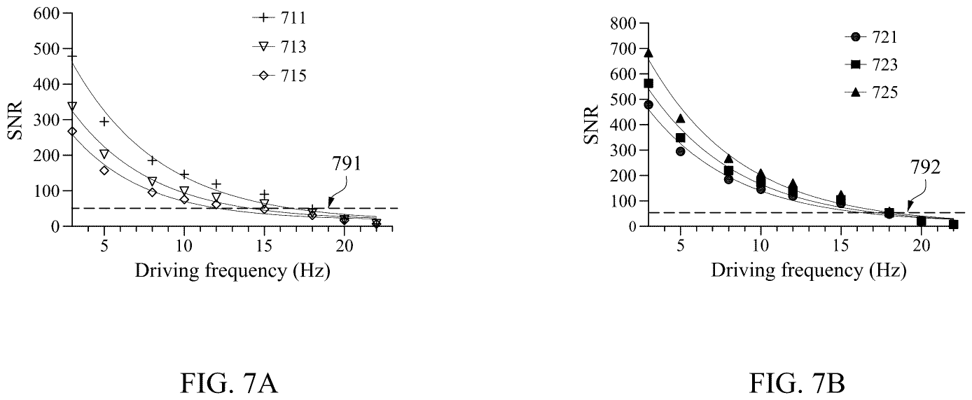

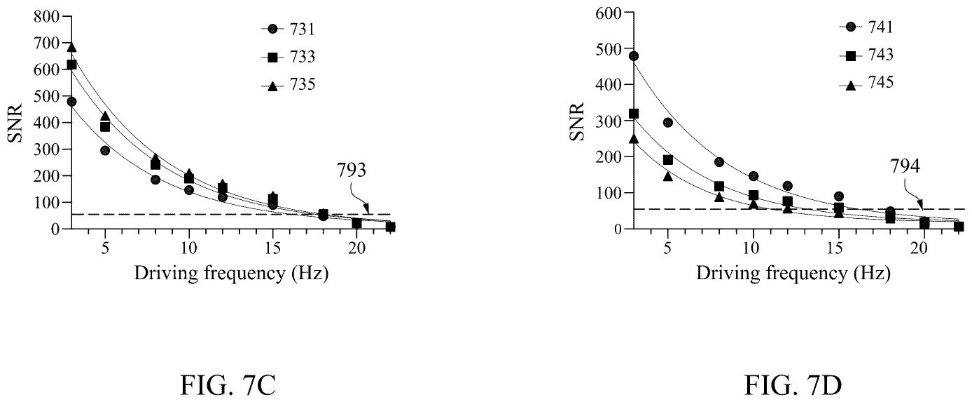

Reference is made to FIGS. 7A to 7D, which illustrate the simulated SNR-driving frequency curved graphs under different viscosities (FIG. 7A), different magnetic field intensities (FIG. 7B), different equivalent particle sizes of the MJP (FIG. 7C), and different particle sizes of the hydrodynamic volumes of the MJP (FIG. 7D). The x-axis represents driving frequencies (unit: Hz), and the y-axis represents SNR. In FIG. 7A, Curve 711, Curve 713, and Curve 715 represent that the viscosities are 1 cP, 2 cP, and 3 cP, respectively. In FIG. 7B, Curve 721, Curve 723, and Curve 725 represent that the magnetic field intensities are 0.010 T, 0.015 T, and 0.030 T, respectively. In FIG. 7C, Curve 731, Curve 733, and Curve 735 represent that the equivalent particle sizes of the MJP are 1 μm, 2 μm, and 3 μm, respectively. In FIG. 7D, Curve 741, Curve 743, and Curve 745 represent that the particle sizes corresponding to the hydrodynamic volumes of the MJP are 1.0 μm, 1.3 μm, and 1.5 μm, respectively.

By calculation, the cut-off frequencies obtained from Curve 711, Curve 713, and Curve 715 are 16.8 Hz, 14.3 Hz, and 12.2 Hz, with an SNR of 2.5 as the standard, respectively, indicating that the bigger the viscosity, the smaller the upper limit of the back-and-forth rotation angle of the MJP, and thus the smaller the cut-off frequency, proving that the upper limit of the back-and-forth rotation angle of the MJP is inversely proportional to the viscosity.

Moreover, the cut-off frequencies obtained from Curve 721, Curve 723, and Curve 725 in FIG. 7B with an SNR of 2.5 as the standard are 16.8 Hz, 17.8 Hz, and 18.7 Hz, respectively, indicating that the bigger the magnetic field intensity, the bigger the upper limit of the back-and-forth rotation angle of the MJP, and thus the bigger the cut-off frequency, indicating that the upper limit of the back-and-forth rotation angle of the MJP is proportional to the magnetic field intensity.

In addition, the cut-off frequencies obtained from Curve 731, Curve 733, and Curve 735 in FIG. 7C are 16.8 Hz, 18.2 Hz, and 18.7 Hz with an SNR of 2.5 as the standard, respectively, indicating that the bigger the equivalent particle size of the MJP, the bigger the upper limit of the back-and-forth rotation angle of the MJP, so that the bigger the cut-off frequency, indicating that the upper limit of the back-and-forth rotation angle of the MJP is positively correlated to the equivalent particle size.

The cut-off frequencies obtained from Curve 741, Curve 743, and Curve 745 in FIG. 7D are 16.8 Hz, 13.8 Hz, and 11.6 Hz with an SNR of 2.5 as the standard, respectively, indicating that the bigger the particle sizes corresponding to the hydrodynamic volume of the MJP, the smaller the upper limit of the back-and-forth rotation angle of the MJP, and the smaller the cut-off frequency, proving that the particle size of the hydrodynamic volume of MJP is inversely proportional to the upper limit of the back-and-forth rotation angle of the MJP.

### Example 2: Estimating SNR-Driving Frequency Curve of MJP at Different Conditions in Practical

1. Preparing MJPs and MJPs Conjugated with Detecting Antibody

After the fluorescent polystyrene (PS hereafter) particles (F13083, Thermo Fisher, MA, USA) were mixed with 95% ethanol and formed an ethanol solution of the fluorescent PS particle, the ethanol solution of the fluorescent PS particles was spread over a glass slide by drop deposition to form a single layer of particles. After drying, an e-beam evaporator was used to deposit a 10 nm silver layer, a 20 nm nickel layer, and an 8 nm gold layer successively under a coating rate of 1 \acute{Å}/S on the hemisphere surface of the fluorescent polystyrene particles away from the glass slide, so as to expose the other hemisphere surface of the fluorescent polystyrene particle.

Then, the glass slide was immersed in a 55 mL centrifuge tube full of deionized water mixed with 0.1 volume % of TWEEN-20. Next, the fluorescent polystyrene particles were collected from the glass slide by sonication, and the solution in the centrifuge tube was filtered with a filter disk (pore size: 3.2 μm) to remove aggregates and metal debris in the solution, thereby obtaining a suspension of MJPs. The concentration of the MJPs in the suspension was adjusted to 2×10⁹ particles/mL.

The suspension was treated with a conjugation reaction with a gold conjugation kit (ab154873, Abcam, Cambridge, UK) so that the CD-63 antibodies were able to conjugate with the gold layer of the MJP via the thiol bond. The method of conjugation reaction was described briefly as follows. A mixed solution was obtained by mixing 0.5 mg/ml, 2.4 μL CD-63 antibodies, 9.6 μL diluted antibody solution, and 42 μL of the gold reaction buffer, and the mixed solution was added to the suspension with the volume ratio of the mixed solution and the suspension as 45 μL:20 μL. Subsequently, the mixed solution and the suspension were incubated in a shaker (rotation speed: 800 rpm) for 20 minutes at room temperature (25° C.±10° C.) and gently mixed with 5 μL of a gold quencher buffer. By the conjugation reaction, the CD-63 antibodies were able to be covalently conjugated on the surface of the particle through lysine residues, thereby forming the magnetic Janus particle conjugated with a detecting antibody (abbreviated as conjugated MJP in the following). The solution in the centrifuge tube was filtered with a syringe filter to obtain a conjugated MJP solution, and the concentration of the conjugated MJP solution was adjusted to 2×10⁹ particles/mL with deionized water. Ideally, when the particle size of the MJP was 1 μm, the number of the antibodies on an MJP could be 15707 presuming that the area of an antibody was 10 nm (equivalent to 10×10⁻⁹ m)×10 nm. Reference was made to Formula (4).

$$\frac{\frac{4 π {R}^{2}}{2}}{( 10 \times 1 {0}^{- 9} )} = 50 \; 00 π ∼ 15708 . \qquad ( 4 )$$

The symbol R represented the radius of the MJP, i.e., the half (10⁻⁶/2 m) of the particle size (1 μm, equivalent to 10⁻⁶ m).

2. Amplitude of Fluorescence Intensity of MJPs without External Magnetic Field

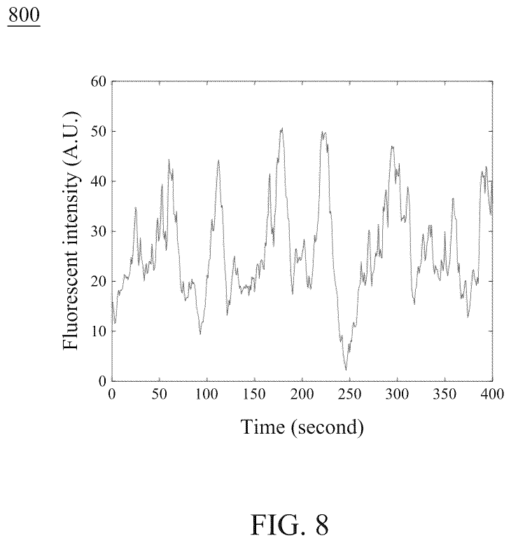

A commercial cameral (FPS was 78 Hz) was used to observe and detect the changes in the fluorescence intensity of the MJPs having an equivalent particle size of 1 μm over time with an excitation light (wavelength was 530 nm to 550 nm) but without N external magnetic field. Reference is made to FIG. 8, which is a curved graph 800 illustrating the changes in the fluorescence intensity of the MJPs over time without an external magnetic field. The x-axis represents time (unit: second), and the y-axis represents the fluorescence intensity change (unit: A.U.). As shown in FIG. 8, the rotation and the movement of the MJPs are random and subjected to Brownian movement without an external magnetic field, and the fluorescence intensity of the MJPs change at 1.42 Hz.

3. Amplitude of Fluorescence Intensity of MJPs Under External Magnetic Field that the Polar was Fixed

The cameral (FPS was 78 Hz) was used to observe and detect the changes in the fluorescence intensities of the MJPs having an equivalent particle size of 1 μm over time under excitation light (wavelength was 530 nm to 550 nm) and an external magnetic field that the driving frequency was 0 Hz (i.e., the polarity was fixed).

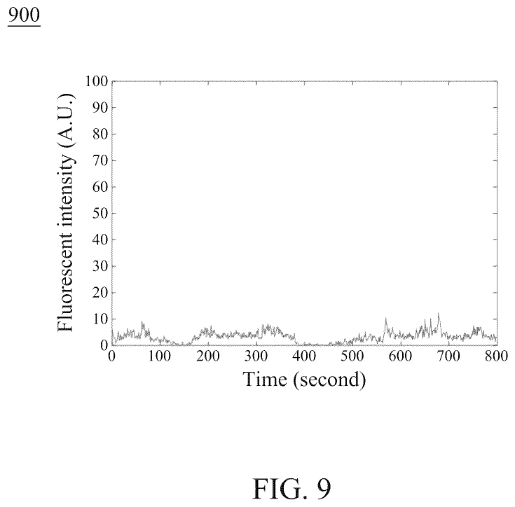

Reference is made to FIG. 9, which is a curved graph 900 illustrating the fluorescence intensity of the MJPs over time under an external magnetic field whose polarity is fixed. The x-axis represents time (unit: second), and the y-axis represents the changes in fluorescence intensity (unit: A.U.). As shown in FIG. 9, the MJPs hardly rotate under the external magnetic field whose polarity is fixed, and thus the amplitude of the fluorescence intensity of the MJPs can hardly be observed, indicating that the external magnetic field is able to control the rotations of the MJPs.

### 4. Amplitude of Fluorescence Intensity of MJPs in Alternative Magnetic Field

The camera (FPS was 80 Hz) was used to observe and detect the change in the fluorescence intensity of the MJPs over time in the testing stage and the driving stage under excitation light (wavelength was 530 nm to 550 nm). During the testing stage, the testing frequency was 1 Hz, and the driving frequencies in the driving stage were 3 Hz, 5 Hz, 8 Hz, 10 Hz, 12 Hz, 15 Hz, 20 Hz, and 22 Hz, respectively. The testing stage and the driving stage were performed successively for 400 seconds. The equivalent particle size of the MJP was 1 μm, the voltage of the electromagnet was 2.7 voltages, and the distance of the electromagnet to the solution was 2.7 cm. In the followings, the driving frequencies of 3 Hz and 22 Hz were used as a representative example for explanation.

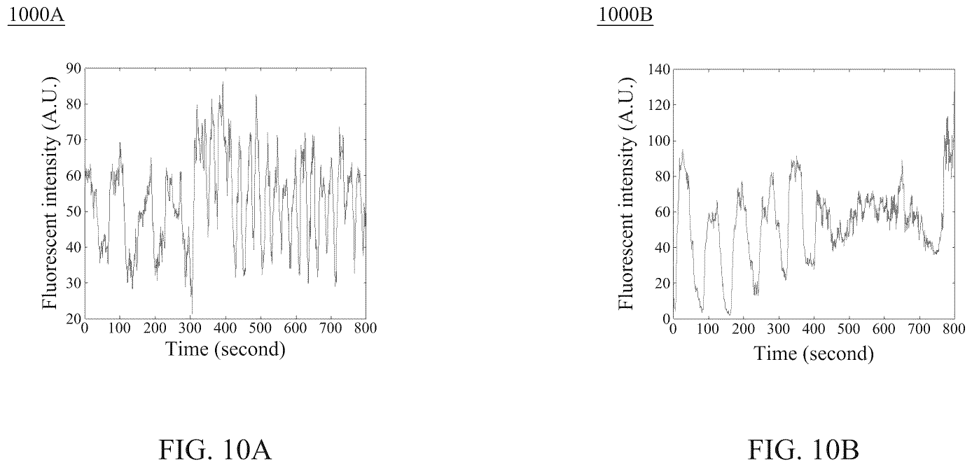

Reference is made to FIGS. 10A and 10B, which are a curved graph 1000A and a curved graph 1000B illustrating the fluorescence intensity of the MJPs over time as each of the driving frequencies is 3 Hz (FIG. 10A) and 22 Hz (FIG. 10B), respectively. The x-axis represents time (unit: second), and the y-axis represents fluorescence intensity (unit: A.U.).

As shown in FIGS. 10A and 10B, the fluorescence intensity of the MJPs changes periodically during the testing stage, indicating that the MJPs rotate back and forth in the alternative magnetic field. Moreover, the MJPs in the alternative magnetic field at a driving frequency of 3 Hz have a bigger amplitude of the fluorescence intensity compared to that of the MJPs in the alternative magnetic field at a driving frequency of 22 Hz, indicating that the higher the driving frequency, the smaller the upper limit of the rotation angle of the MJPs, and the smaller the change in the area of the hemisphere surface of the fluorescent PS particle that was exposed and projected to the observation direction, and thus the smaller the amplitude of the fluorescence intensity detected in the observation direction.

The SNR could be obtained by calculating the average of the ratios of the upper limits of the amplitudes of the fluorescence intensities of the MJPs to the background fluorescence intensities. An SNR-driving frequency curve could be illustrated according to the SNRs at a plurality of driving frequencies. It is noted that due to the unideal rotation direction of some MJPs, the unideal magnetization of some MJPs, or other factors, the fluorescence intensity might not change with the polarity change of the alternative magnetic field, thereby negatively affecting the subsequent analyses. Therefore, by performing the testing stage with a lower testing frequency beforehand, the fluorescence intensity of the MJPs under observation could be ensured to change corresponding to the alternative magnetic field.

### Example 3: Estimating SNR-Driving Frequency Curve and Cut-Off Frequency of MJPs Under Different Conditions Practically

Unless otherwise specified, the steps described below are performed under the following conditions: the viscosity of the solution was 7.06 cP; the distance between the electromagnet and the solution was 2.7 cm; the voltage amplitude introduced to the electromagnet was 0.2 V; the equivalent particle size of the MJPs was 1 μm; and the measurement was performed at room temperature (25±10° C.). The cut-off frequency was obtained from the SNR-driving frequency curve with an SNR of 2.5 as the standard.

1. SNR-Driving Frequency Curves and Cut-Off Frequencies of MJPs with Different Equivalent Particle Sizes

The SNR-driving frequency curves of the MJPs with equivalent particle sizes of 1 μm, 2 μm, and 3 μm were obtained by detecting the SNRs of the MJPs with equivalent particle sizes of 1 μm, 2 μm, and 3 μm corresponding to different driving frequencies of the alternative magnetic fields.

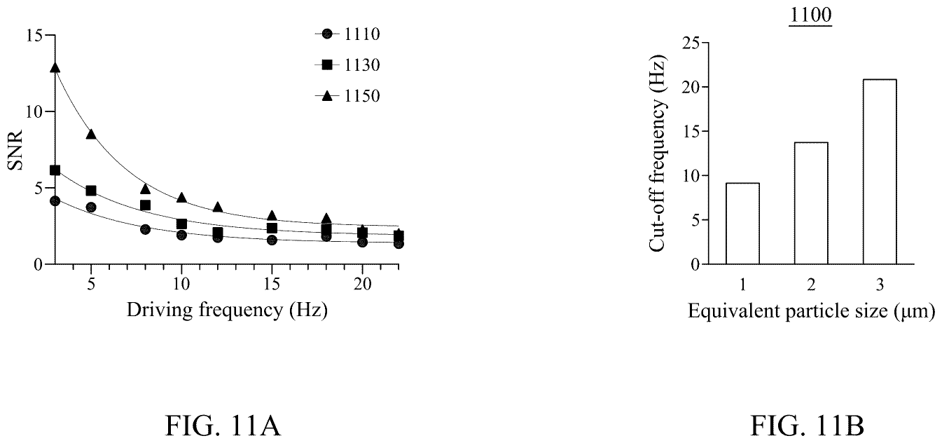

Reference is made to FIG. 11A, which illustrates an SNR-driving frequency curved graph of the MJPs with different equivalent particle sizes according to some embodiments of the present invention, in which Curve 1110 represents the MJPs having an equivalent particle size of 1, Curve 1130 represents the MJP having an equivalent particle size of 2, and Curve 1150 represents the MJP having an equivalent particle size of 3.

Reference is made to FIG. 11B, which is a bar graph 1100 illustrating the cut-off frequencies of the SNR-driving frequency curves of the MJPs having different equivalent particle sizes according to the SNR-driving frequency curve in FIG. 11A. As shown in FIG. 11B, when the equivalent particle sizes of the MJPs are 1 μm, 2 μm, and 3 μm, the cut-off frequencies are 9.12 Hz, 13.71 Hz, and 20.83 Hz, respectively, indicating the bigger the equivalent particle size of the MJPs, the bigger the cut-off frequency of the SNR-driving frequency curve.

As shown in Formulas (1) and (2), the imaginary part of the magnetic susceptibility and the volume of the magnetic material are proportional to the upper limit of the back-and-forth rotation angle of the MJPs. Moreover, the imaginary part of the magnetic susceptibility, the volume of the magnetic material, and the upper limit of the fluorescence intensity changes of the MJPs are proportional to the square of the equivalent particle size of the MJPs. Therefore, the bigger the equivalent particle size of the MJPs, the bigger the cut-off frequency. The results of FIGS. 11A and 11B comply with Formulas (1) and (2).

### 2. SNR-Driving Frequency Curve and Cut-Off Frequency of MJPs at Different Voltage Amplitudes

The SNR-driving frequency curves of the MJPs at voltage amplitudes of 0.2 V and 0.4 V were obtained by detecting the SNRs of the MJPs corresponding to different driving frequencies when voltage amplitudes of 0.2 V and 0.4 V were introduced to the electromagnet generating the alternative magnetic field, respectively.

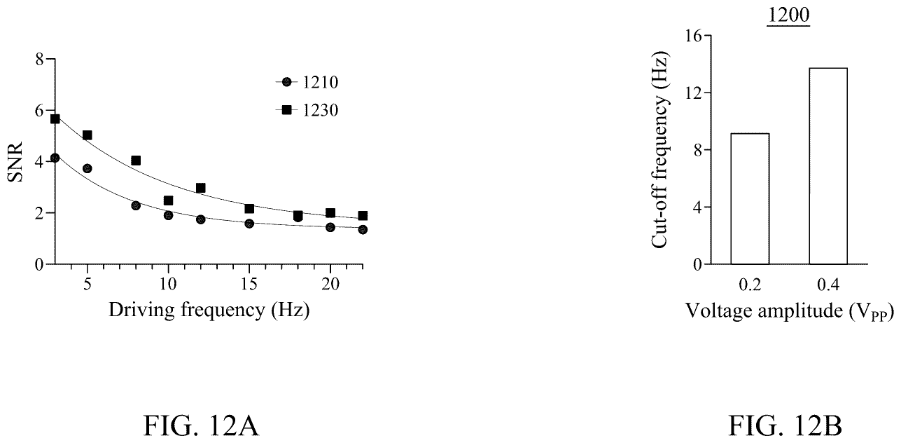

Reference is made to FIG. 12A, which illustrates an SNR-driving frequency curved graph of the MJPs at different voltage amplitudes according to some embodiments of the present invention. Curve 1210 represents a voltage amplitude of 0.2 V, and Curve 1230 represents a voltage amplitude of 0.4 V.

Reference is made to FIG. 12B, which is a bar graph 1200 illustrating the cut-off frequencies of the SNR-driving frequency curves of the MJPs at different voltage amplitudes according to the SNR-driving frequency curve in FIG. 12A. As shown in FIG. 12B, when the voltage amplitudes are 0.2 V and 0.4 V, the cut-off frequencies of the SNR-driving frequency curves of the MJPs are 9.12 Hz and 13.71 Hz, respectively, indicating that the bigger the voltage amplitude introduced to the electromagnet, the bigger the cut-off frequency of the SNR-driving frequency curve of the MJPs.

As shown in Formulas (1) and (2), the magnetic field intensity is proportional to the upper limit of the back-and-forth rotation angle of the MJPs. Moreover, the magnetic field intensity is proportional to the voltage amplitude introduced to the electromagnet. Thus, the bigger the voltage amplitude introduced to the electromagnet, the bigger the cut-off frequency. The results of FIGS. 12A and 12B can comply with Formulas (1) and (2).

3. SNR-Driving Frequency Curves and Cut-Off Frequencies of MJPs with Different Distances Away from Electromagnet

The SNR-driving frequency curves of the MJPs with distances of 2.7 cm and 3.6 cm away from the electromagnet were obtained by detecting the SNRs of the MJPs with distances of 2.7 cm and 3.6 cm away from the electromagnet, respectively.

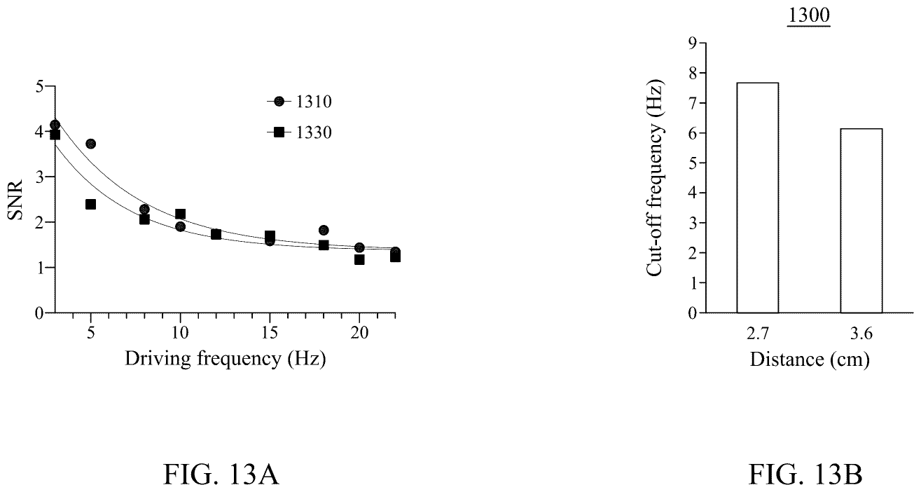

Reference is made to FIG. 13A, which illustrates an SNR-driving frequency curved graph of the MJPs with different distances away from the electromagnet according to some embodiments of the present invention. Curve 1310 represents a distance of 2.7 cm away from the electromagnet, and Curve 1330 represents a distance of 3.6 cm away from the electromagnet.

Reference is made to FIG. 13B, which is a bar graph 1300 illustrating the cut-off frequency of the SNR-driving frequency curve of the MJPs with different distances away from the electromagnet according to FIG. 13A. As shown in FIG. 13B, the bigger the distance away from the electromagnet, the smaller the cut-off frequencies of the SNR-driving frequency curve of the MJPs.

As shown in Formulas (1) and (2), the magnetic field intensity is proportional to the upper limit of the back-and-forth rotation angle of the MJPs. However, the magnetic field intensity is inversely proportional to the distance between the solution and the electromagnet. Therefore, the bigger the distance between the solution and the electromagnet, the smaller the cut-off frequency. The results of FIGS. 13A and 13B can comply with Formulas (1) and (2).

### 4. SNR-Driving Frequency Curve and Cut-Off Frequency of MJPs at Different Viscosities

The SNR-driving frequency curves of the MJPs at the viscosities of 0.98 cP, 1.52 cP, 2.31 cP, and 7.06 cP were obtained, respectively, by detecting the SNRs of the MJPs corresponding to different driving frequencies at the viscosities of 0.98 cP, 1.52 cP, 2.31 cP, and 7.06 cP.

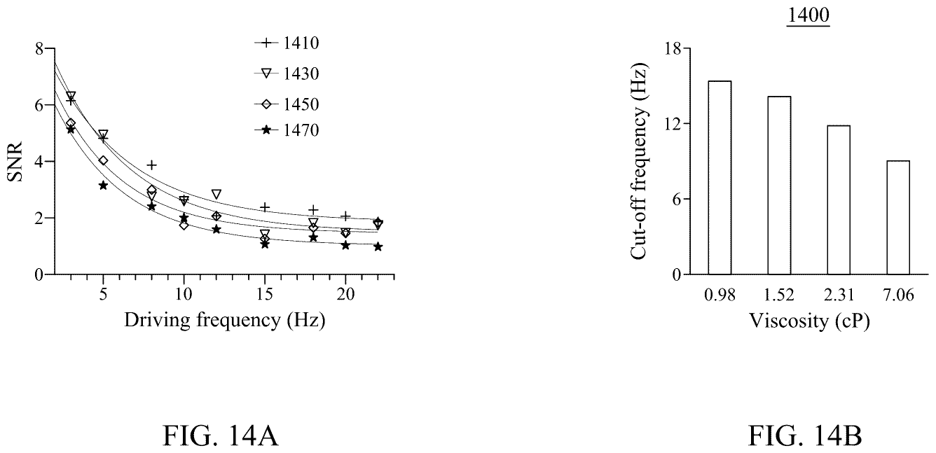

Reference is made to FIG. 14A, which illustrates an SNR-driving frequency curved graph of the MJPs at different viscosities according to some embodiments of the present invention. Curve 1410 represents a viscosity of 0.98 cP, Curve 1430 represents a viscosity of 1.52 cP, Curve 1450 represents a viscosity of 2.31 cP, and Curve 1470 represents a viscosity of 7.06 cP.

Reference is made to FIG. 14B, which is a bar graph 1400 illustrating the cut-off frequency of the SNR-driving frequency curve of the MJPs at different viscosities according to FIG. 14. As shown in FIG. 14B, when the viscosities are 0.98 cP, 1.52 cP, 2.31 cP, and 7.06 cP, the cut-off frequencies of the SNR-driving frequency curves of the MJPs are 15.36 Hz, 14.13 Hz, 11.81 Hz, and 9.01 Hz, respectively, indicating that the higher the viscosity, the smaller the cut-off frequency of the SNR-driving frequency curve. It is noted that the viscosity is closely inversely proportional to the cut-off frequency, proving that the method can be used to detect not only the exosome concentration but also the parameters such as viscosity.

As shown in Formulas (1) and (2), the viscosity is inversely proportional to the upper limit of the rotation angle of the MJPs. Thus, when MJPs are in a solution with a higher viscosity, the SNR-driving frequency curve of the MJPs moves to the left, and the cut-off frequency will be smaller. The results shown in FIGS. 14A and 14B can comply with Formulas (1) and (2).

### Example 4: Determine Whether the Testing Method can be Applied to Detecting Exosome Concentration in Test Sample Practically

### 1. Exosome Isolation Treatment

Human oral squamous carcinoma cell line OECM-1 (referred to as OECM-1 strain hereafter) was incubated in a culture plated at 37° C. The medium used to incubate the OECM-1 strain included 10 volume % fetal bovine serum, 1 volume % antibiotic, and 10 mL Roswell Park Memorial Institute (RPMI hereafter) medium. The medium was replaced with a 10 mL RPMI medium containing 1 volume % antibiotic without fetal bovine serum.

The culture medium was collected and centrifuged at 900×g for 30 minutes to pellet the cell debris, and the supernatant obtained from the culture medium subjected to centrifugation was filtered with a 0.22 μm syringe filter to remove the molecules with diameters bigger than 0.22 μm, thereby obtaining a cell-free filtrate.

Then, the cell-free filtrate was subjected to ultracentrifugation at a rotation speed of 100,000×g for 4 hours, so that the exosome formed a pellet. The pellet was re-suspended with 1×PBS, and after an overnight incubation at 4° C., ultracentrifugation was performed at 4° C., 100,000×g for 2 hours. The pellet was re-suspended by using 1×PBS to obtain an exosome sample. The exosome sample was stored at −80° C. after the preparation. The exosome concentration of the sample was adjusted to 2.9×10¹¹±4.84×10⁹ exosomes/mL with deionized water.

### 2. SNR-Driving Frequency Curve and Cut-Off Frequency of Conjugated MJPs Added in Different Solutions

The conjugated MJPs were added to PBS, the 100× dilution of the exosome sample, the 10× dilution of the exosome sample, the exosome sample, and bovine serum albumin (BSA hereafter), followed by a reaction step at 700 rpm, room temperature for 30 minutes to obtain Comparative Group 1, Example Group 1, Example Group 2, Example Group 3, and Comparative Group 2. The upper limit of the amplitude of the fluorescence intensity of Comparative Group 1, Example Group 1, Example Group 2, Example Group 3, and Comparative Group 2 under an alternative magnetic field at different driving frequencies were detected by the method mentioned above to obtain the SNR-driving frequency curves and the cut-off frequency of the conjugated MJPs in Comparative Group 1, Example Group 1, Example Group 2, Example Group 3, and Comparative Group 2.

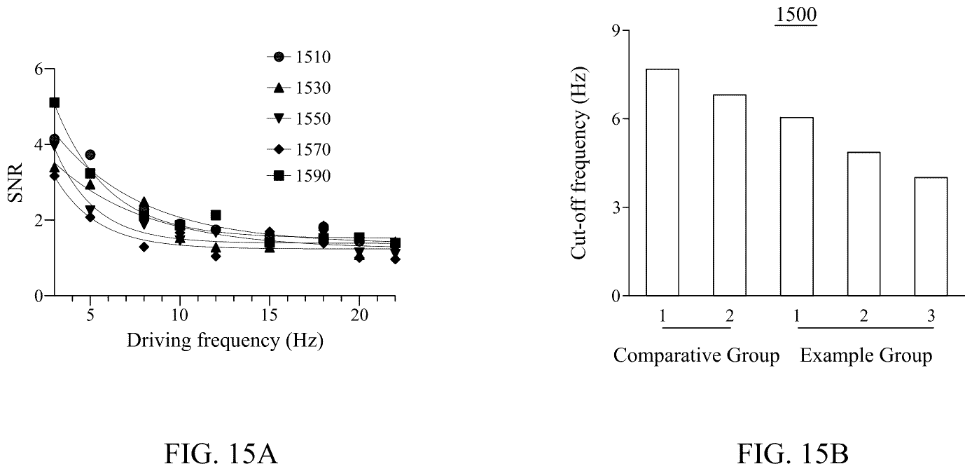

FIG. 15A illustrates the SNR-driving frequency curves in different groups according to different groups of the present invention, in which Curve 1510, Curve 1530, Curve 1550, Curve 1570, and Curve 1590 are the SNR-driving frequency curves of Comparative Group 1, Example Group 1, Example Group 2, Example Group 3, and Comparative Group 2, respectively. The individual cut-off frequencies are obtained from the SNR-driving frequency curves of the Comparative Group 1 (the conjugated MJPs are in the PBS), Example Group 1 (the conjugated MJPs are in the exosome sample), Example Group 2 (the conjugated MJPs are in the 10× dilution of the exosome sample), Example Group 3 (the conjugated MJPs are in the 100× dilution of the exosome sample), and Comparative Group 2 (the conjugated MJPs are in BSA) are obtained with an SNR of 2.5 as the standard.

FIG. 15B is a bar chart 1500 illustrating the cut-off frequencies of the SNR-driving frequency curves in different groups according to FIG. 15A. The x-axis represents the groups, and the y-axis represents the cut-off frequency. The cut-off frequencies in Comparative Group 1, Comparative Group 2, Example Group 1, Example Group 2, and Example Group 3 are 7.68 Hz, 6.81 Hz, 6.04 Hz, 4.86 Hz, and 4.00 Hz, respectively.

As shown in FIGS. 15A and 15B, there is no exosome in the PBS in Comparative Group 1, and thus the cut-off frequency is higher. The BSA included in Comparative Group 2 has a higher viscosity, resulting in a decrease in the cut-off frequency.

The exosome concentration of Example Group 1, Example Group 2, and Example Group 3 increased proportionally so that the hydrodynamic volume of the complex formed by a conjugated MJP and exosomes increased. As shown in Formulas (1) and (2), the hydrodynamic volume of the complex is inversely proportional to the upper limit of the rotation angle of the complex. Thus, the exosome sample with a higher exosome concentration has a lower cut-off frequency obtained by the aforementioned method. The results in FIGS. 15A and 15B comply with Formulas (1) and (2), indicating that the method can be applied to comparing the exosome concentration between the test sample and the standard sample, thereby determining whether the test sample has the risk of early-stage cancer. Thus, the method has the potential to facilitate anticipating whether the sample has the risk of early-stage cancer. It can be seen from the results that the sensitivity of the method is high and can be 2×10⁹ exosomes/mL (Example Group 1). Moreover, since the cut-off frequency of Example Group 1 is different from that of Comparative Group 1, the detection limit can be lower, e.g., 107 exosomes/mL to 108 exosomes/mL.

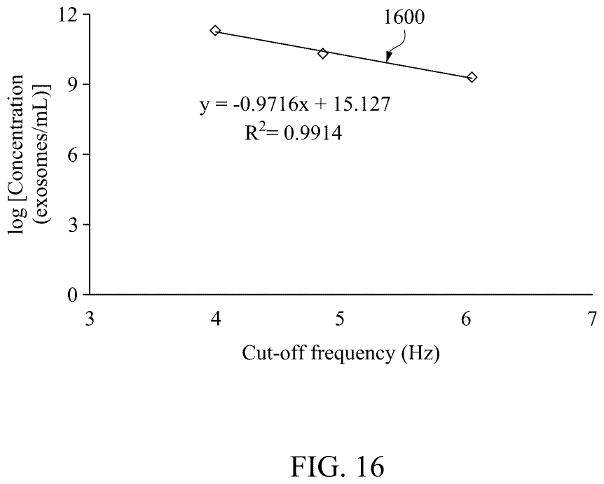

FIG. 16 is a scatter graph illustrating the driving frequencies corresponding to the logarithm of the exosome concentration of Example Groups 1 to 3 according to FIG. 15B. The x-axis represents the driving frequency (unit: Hz), the y-axis represents the logarithm of the concentration (the concentration took exosomes/mL as unit), and Line 1600 represents a regression line obtained by using a linear regression analysis. As shown in FIG. 16, the R² of Line 1600 is 0.9917, indicating that the relationship of the cut-off frequency and the exosome concentration can be fit in a line. The test concentration of the exosome in the test sample can be obtained by performing interpolation and/or extrapolation on Line 1600 used as the standard curve.

In sum, although the specific MJP, the specific driving frequency, the specific distance from the electromagnet, the specific viscosity, the specific origin of the sample, the specific process of manufacturing, or the specific evaluation method are shown in the present invention as examples to explain a method of detecting a cancer biomarker concentration of an in vitro biological sample with a magnetic Janus particle, it will be apparent to those skilled in the art that the present invention is not limited to what have mentioned. Without departing from the scope or spirit of the invention, it is intended that other MJP, other driving frequency, other distance from the electromagnet, other viscosity, other sample, other process of manufacturing, or other evaluation method can also explain the method of detecting a cancer biomarker concentration of an in vitro biological sample with a magnetic Janus particle of present invention.

From the abovementioned embodiment, the method of detecting a cancer biomarker concentration of an in vitro biological sample with a magnetic Janus particle of the present invention uses a magnetic Janus particle and an alternative magnetic field at a plurality of driving frequencies are used to obtain a cut-off frequency. The test concentration is obtained from the cut-off frequency of the test sample according to the standard curve established by using a known concentration of a standard sample and the cut-off frequency. The method can shorten the detecting time as well as decrease the lower limit of the volume and/or the concentration of the sample, thereby increasing the detecting concentration range.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A method of detecting a cancer biomarker concentration of an in vitro biological sample with a magnetic Janus particle conjugated with a detecting antibody, comprising:
providing a solution, wherein the solution comprises the magnetic Janus particle conjugated with the detecting antibody, the magnetic Janus particle comprises:
a fluorescence core; and
a metal hemisphere shell covering a hemisphere surface of the fluorescence core, and the metal hemisphere shell comprising a nickel layer;
wherein the detecting antibody conjugates on a surface of the metal hemisphere shell, and a number ratio of the detecting antibody and the magnetic Janus particle is (150 to 1500000):1;
adding the in vitro biological sample to the solution, wherein the in vitro biological sample comprises a cancer biomarker, the detecting antibody binds to the cancer biomarker, the magnetic Janus particle conjugated with the detecting antibody forms a complex, the in vitro biological sample is a standard sample comprising the cancer biomarker with a known concentration or a test sample originating from a test subject, and the cancer biomarker comprises an exosome;
performing a treatment on the complex to obtain at least one signal-to-noise ratio (SNR)-driving frequency curve of the complex, wherein the treatment comprises applying an excited light and an alternative magnetic field at a plurality of driving frequencies to the complex, a magnetic field intensity of the alternative magnetic field is 0.1 mG to 500 mG, and each of the plurality of the driving frequencies is bigger than 1 Hz to 50 Hz, wherein the at least one SNR-driving frequency curve obtained from the standard sample is a first SNR-driving frequency curve, and the at least one SNR-driving frequency curve obtained from the test sample is a second SNR-driving frequency curve;
obtaining a first cut-off frequency and a second cut-off frequency from the first SNR-driving frequency curve and the second SNR-driving frequency curve, respectively, with an SNR of bigger than 1 and smaller than 5 as a standard; and
obtaining a test concentration of the test sample from the second cut-off frequency according to a standard curve established by the first cut-off frequency corresponding to the known concentration.

2. The method of detecting the cancer biomarker concentration of the in vitro biological sample with the magnetic Janus particle conjugated with the detecting antibody of claim 1, wherein the cancer biomarker is used to facilitate anticipating a cancer, and the cancer comprises at least one of an oral cavity cancer, a colon cancer, a pancreatic cancer, a renal cancer, a bladder cancer, a breast cancer, a prostate cancer, a blood cancer, and a cartilage tumor.

3. The method of detecting the cancer biomarker concentration of the in vitro biological sample with the magnetic Janus particle conjugated with the detecting antibody of claim 1, wherein the in vitro biological sample originates from at least one of a saliva, an oral mucosa, an intestinal juice, a urine, a breast milk, a semen, blood, a tissue fluid, and a synovial fluid.

4. The method of detecting the cancer biomarker concentration of the in vitro biological sample with the magnetic Janus particle conjugated with the detecting antibody of claim 1, wherein the in vitro biological sample is obtained by subjecting an exosome isolation treatment.

5. The method of detecting the cancer biomarker concentration of the in vitro biological sample with the magnetic Janus particle conjugated with the detecting antibody of claim 1, wherein the detecting antibody comprises at least one of a CD63 antibody, a CD44 antibody, a CD81 antibody, an ADAM10 antibody, and an MMP14 antibody.

6. The method of detecting the cancer biomarker concentration of the in vitro biological sample with the magnetic Janus particle conjugated with the detecting antibody of claim 1, wherein the metal hemisphere shell further comprises a silver layer, and the silver layer is disposed between the nickel layer and the hemisphere surface of the fluorescence core.

7. The method of detecting the cancer biomarker concentration of the in vitro biological sample with the magnetic Janus particle conjugated with the detecting antibody of claim 1, wherein the metal hemisphere shell further comprises a gold layer disposed on the nickel layer.

8. The method of detecting the cancer biomarker concentration of the in vitro biological sample with the magnetic Janus particle conjugated with the detecting antibody of claim 1, wherein a wavelength of the excitation light is 530 nm to 550 nm.

9. The method of detecting the cancer biomarker concentration of the in vitro biological sample with the magnetic Janus particle conjugated with the detecting antibody of claim 1, further comprising:
performing a pre-treatment on the complex before the treatment, wherein the pre-treatment comprises applying the excitation light and the alternative magnetic field at a testing frequency to the complex, and the testing frequency is smaller than each of the plurality of driving frequencies.

10. The method of detecting the cancer biomarker concentration of the in vitro biological sample with the magnetic Janus particle conjugated with the detecting antibody of claim 1, further comprises:
performing a reaction step after the in vitro biological sample is added to the solution at 500 rpm to 1000 rpm for 20 minutes to 60 minutes.

11. The method of detecting the cancer biomarker concentration of the in vitro biological sample with the magnetic Janus particle conjugated with the detecting antibody of claim 1, wherein the standard curve is obtained by a linear regression analysis.

12. The method of detecting the cancer biomarker concentration of the in vitro biological sample with the magnetic Janus particle conjugated with the detecting antibody of claim 1, wherein the standard sample comprises a standard stock solution and a standard diluted solution obtained by diluting the standard stock solution at a specific dilution ratio.

13. The method of detecting the cancer biomarker concentration of the in vitro biological sample with the magnetic Janus particle conjugated with the detecting antibody of claim 1, wherein an area of the hemisphere surface covered by the metal hemisphere shell accounts for 40% to 60% of a surface of the fluorescence core.

14. A method of detecting an exosome concentration of an in vitro biological sample with a magnetic Janus particle conjugated with a detecting antibody, comprising:
providing a solution, wherein the solution comprises the magnetic Janus particle conjugated with the detecting antibody, and the magnetic Janus particle comprises:
a fluorescence core; and
a metal hemisphere shell covering a hemisphere surface of the fluorescence core, the metal hemisphere shell comprising a silver layer, a nickel layer covering the silver layer, and a gold layer covering the nickel layer;
wherein the detecting antibody conjugates on a surface of the metal hemisphere shell, wherein a number ratio of the detecting antibody and the magnetic Janus particle is (150 to 1500000):1;
adding the in vitro biological sample to the solution, wherein the in vitro biological sample comprises an exosome, the detecting antibody binds to a transmembrane protein on the exosome, the magnetic Janus particle conjugated with the detecting antibody forms a complex, wherein the in vitro biological sample is a standard sample comprising the exosome with a known concentration or a test sample originating from a test subject;
performing a treatment on the complex to obtain at least one SNR-driving frequency curve of the complex, wherein the treatment comprises applying an excited light and an alternative magnetic field at a plurality of driving frequencies to the complex, a magnetic field intensity of the alternative magnetic field is 0.1 mG to 500 mG, and each of the plurality of the driving frequencies is bigger than 1 Hz to 50 Hz, wherein the at least one SNR-driving frequency curve obtained from the at least one standard sample is a first SNR-driving frequency curve, and the SNR-driving frequency curve obtained from the test sample is a second SNR-driving frequency curve;
obtaining a first cut-off frequency and a second cut-off frequency from the first SNR-driving frequency curve and the second SNR-driving frequency curve, respectively, with an SNR of bigger than 1 and smaller than 5 as a standard; and
establishing a standard curve by the first cut-off frequency corresponding to the known concentration, and a test concentration of the test sample is obtained from the second cut-off frequency according to the standard curve.

15. The method of detecting the exosome concentration of the in vitro biological sample with the magnetic Janus particle conjugated with the detecting antibody of claim 14, wherein the transmembrane protein comprises a CD63, a CD44, a CD81, an ADAM10, and an MMP14.
